(19) United States
(12) Patent Application Publication
(10) Pub. No.: US 2013/0152933 A1
(43) Pub. Date: Jun. 20, 2013

(54) AUTOMATED FLUID DELIVERY SYSTEM AND METHOD

(71) Applicants: David William Lischer, Carlsbad, CA (US); Stephen Winston Roberts, La Jolla, CA (US)

(72) Inventors: David William Lischer, Carlsbad, CA (US); Stephen Winston Roberts, La Jolla, CA (US)

(73) Assignee: THE REGENTS OF THE UNIVERSITY OF CALIFORNIA, A CALIFORNIA CORPORATION, OAKLAND, CA (US)

(21) Appl. No.: 13/814,934

(22) PCT Filed: Aug. 9, 2011
(86) PCT No.: PCT/US2011/047148

§ 371 Date: Feb. 7, 2013

Related U.S. Application Data
(60) Provisional application No. 61/372,411, filed on Aug. 10, 2010.

(51) Int. Cl. A61M 16/08; A61M 16/20; A61M 16/00

(52) U.S. Cl. CPC A61M 16/0875; A61M 16/0057; A61M 16/20
USPC 128/204.23

(57) ABSTRACT

An automated fluid delivery system and method are disclosed. The system includes distensible tubing, a flow controller, and a fluid flow adjustment module. The fluid flow adjustment module may be configured to detect differential pressure in the tubing and adjust the flow controller to provide an amount of fluid through the tubing during inhalation.

### CROSS-REFERENCE TO RELATED APPLICATION

This application claims the benefit of priority of U.S. provisional patent application No. 61/372,411, filed, Aug. 10, 2010, the contents of which are incorporated herein by reference

### BACKGROUND OF THE INVENTION

The present invention generally relates to fluid systems, and more particularly, to an automated fluid delivery system.

Some individuals benefit from the use of supplemental fluid delivery systems. For example, a person with chronic obstructive pulmonary disease (COPD), or other lung insufficiency, may need supplemental oxygen, which is commonly sourced from a compressed oxygen cylinder, to maintain a physiologically adequate degree of oxygen saturation in the blood. Supplemental oxygen delivery typically involves a tubing connection to a tank and a pressure regulator for an extended period of time. Others, for example, athletes, aircraft pilots, travelers at mountainous high altitudes, may need temporary oxygen supplementation because of exertion or low ambient oxygen.

Some conventional fluid delivery systems provide a predetermined flow of oxygen to the end user. A conventional system typically requires manual adjustment of a valve in a pressure regulator attached to a cylinder of compressed oxygen. The flow rate of oxygen provided is predetermined and often remains unadjusted while the system is in use. Typically, the flow rate of oxygen provided is overestimated to avoid undersupplying oxygen to the user. However, this is wasteful of the oxygen.

Other systems, known as oxygen conserver systems, deliver oxygen to users in pulses. The length and amplitude of the pulses are manually determined by setting a rotary switch. Thus, the amount of oxygen per pulse remains constant until the switch is re-adjusted.

It is also known to deliver an oxygen pulse to a user based on tracking the user's breathing frequency and automatically adjusting the amount of oxygen delivered based on repetition rate of past breaths. This technique relies on past data to predict what quantity of oxygen future breaths will require.

As can be seen, there is a need for a system and method that may provide an immediate optimum amount of fluid based on real-time need while minimizing unnecessary expenditure of oxygen

### SUMMARY OF THE INVENTION

In one aspect of the present invention, a system of providing fluid to a user comprises distensible tubing, a flow controller coupled to the tubing and configured to control a flow of fluid through the tubing, and a fluid flow adjustment module connected to the tubing and the flow controller. The module is configured to measure pressure changes in the tubing during a single inhalation and to control the flow controller to provide an optimum amount of the fluid through the tubing based on the measured pressure changes during the inhalation.

In another aspect of the present invention, a system of providing oxygen to a user comprises distensible tubing connected between an oxygen source and the user to provide an amount of oxygen to the user, a flow controller coupled to the tubing and configured to control the amount of oxygen through the tubing a pressure sensor connected to the tubing between the flow controller and the user and a microcontroller coupled to the pressure sensor. The microcontroller is configured to receive pressure signals provided by the pressure sensor, detect the start of a breathing event from the user based on a first pressure signal, determine an amount of oxygen needed by the user based on a second pressure signal, and control the flow controller to adjust the amount of oxygen flow to the user based on the second pressure signal. The pressure signals are detected from differential pressure in the tubing.

In still yet another aspect, a method of providing oxygen to a user may include detecting the start of a first breathing event in tubing connected to the user, analyzing a magnitude of pressure change in the tubing during a predetermined time frame, determining an amount of oxygen needed by the user during the first breathing event based on the magnitude of pressure change analyzed, and supplying the determined amount of oxygen to the user.

These and other features, aspects and advantages of the present invention will become better understood with reference to the following drawings, description and claims.

### BRIEF DESCRIPTION OF THE DRAWINGS

FIG. 1 is a block diagram illustrating an automated oxygen delivery system according an exemplary embodiment of the present invention;

FIG. 2 is a schematic diagram of a circuit according an exemplary embodiment of the present invention;

FIG. 3 is a flow diagram of steps in a method according an exemplary embodiment of the present invention; and

FIG. 4 is a plot illustrating a timeline of a breathing event according an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features. However, any single inventive feature may not address any of the problems discussed above or may only address one of the problems discussed above. Further, one or more of the problems discussed above may not be fully addressed by any of the features described below.

Broadly, embodiments of the present invention generally may provide an automated system adapted to provide an optimum bolus of oxygen based on measured needs of a user. In one aspect, the system may supply supplemental oxygen to a human or other animal on an as-needed basis of a breathing event, also referred to as a breath cycle. A breath cycle may include an inhalation phase and an exhalation phase. The oxygen need may be estimated on a breath-by-breath basis by measuring and analyzing pressure characteristics of each breath. Oxygen flow requirements to meet the oxygen need may then be predicted (e.g., calculated) by a microcontroller. An oxygen bolus may then be produced, appropriate in timing and amount, to meet the current need during a detected inhalation. Thus, in one aspect, upon detection of an inhalation, an optimum amount of fluid may be supplied during the same detected inhalation. The system may be dynamic and continuously responsive to the varying oxygen need of a user.

In one possible embodiment, it may be desirable to maintain the oxygen blood saturation level within a physiologically appropriately range. The flow of oxygen may be adjusted based on real-time measurements by a blood oxygen sensor. One such sensor may be a pulse oximeter. The oximeter input may be used in combination with the inhalation pressure measurement technique described in the disclosure that follows.

In another aspect, oxygen need may be determined by measuring the carbon dioxide level of each exhalation. Such a measurement may be useful in a hospital setting for example, where accurate monitoring of a patient is desirable.

In some possible embodiments, the system may be battery powered and portable, with some elements assembled onto a circuit board for facilitated plug and play connection to a user and a portable fluid source.

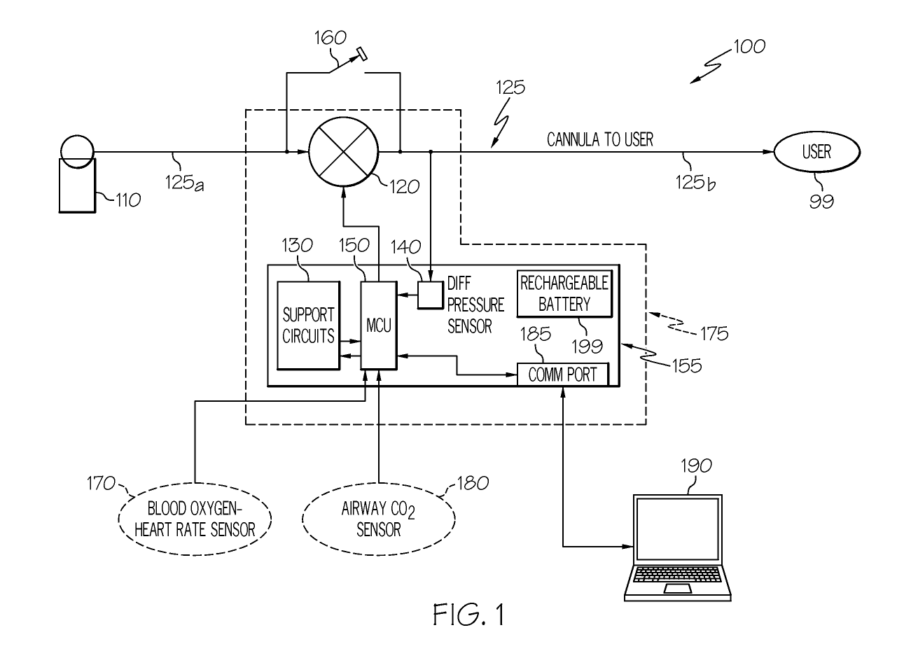

Referring to FIG. 1, an automated system 100, (also referred to in general as the system) of providing oxygen to a user 99 is shown. The system 100 includes a flow controller 120, tubing 125, and a fluid flow adjustment module 175. Power to the system 100 may be provided by a power source 199. The power source 199 may be, for example, a rechargeable battery. However, while the power source 199 is shown as coupled directly to the fluid flow adjustment module 175, it will be understood that other exemplary embodiments may include power sources 199 disposed externally to the module 175, for example, by use of a conventional transformer plugged into a wall outlet.

In an exemplary embodiment, the tubing 125 may be connected to a regulated fluid source 110 and configured to deliver fluid to the user 99. The tubing 125 may be distensible tubing, for example a cannula. The fluid source 110 may be, for example, a small portable cylinder of compressed oxygen, as ordinarily used in other supplemental oxygen systems. The flow controller 120 may be coupled to the tubing 125 and disposed between a first tubing segment 125a and a second tubing segment 125b. The flow controller 120 may include (not shown) one or more on/off pneumatic flow valves, a proportional flow valve, a mass flow controller, or some other device to control fluid flow in response to an electronic control signal. The first tubing segment 125a may be disposed between the fluid source 110 and the flow controller 120. The second tubing segment 125b may be disposed between the flow controller 120 and the user 99. A bypass valve 160 may also be connected between tubing segments 125a and 125b, and during normal operation of the system 100, configured to prohibit the flow of fluid around the flow controller 120. In the event of a malfunction of the automated system 100, fluid may be prevented from passing from oxygen source 110 to the user 99. The bypass valve 160 may then be manually switched on thus providing a secondary flow path to the user 99.

The fluid flow adjustment module 175 may be coupled to the flow controller 120 and the second tubing segment 125b. In an exemplary embodiment, the fluid adjustment module 175 may include a pressure sensor 140, a microcontroller 150, a blood oxygen sensor 170, and a carbon dioxide sensor 180. In some embodiments, the fluid flow adjustment module 175 may also include a communications port 185 for connection to a monitoring device/communications device 190, for example a personal computer or data recorder. The microcontroller 150, pressure sensor 140, communications port 185, and a plurality of support circuits 130 may be assembled onto a circuit board assembly 155.

The microcontroller 150 determines and controls the amount of fluid administered to the user 99. The microcontroller 150 may be connected to the flow controller 120. The microcontroller 150 may be, for example, a model Microchip PIC 16F88. The microcontroller 150 may be configured to store operating software that controls measurement of pressure and other system data, and commands the flow controller 120 to supply an optimum amount of fluid as needed. The microcontroller 150 may also be connected to the pressure sensor 140.

The microcontroller 150 may continuously analyze electrical output from the pressure sensor 140 for the detection of a breathing event and for the calculation of an optimum amount of fluid that should be supplied to the user 99. The pressure sensor 140 may be configured to continuously sense pressure magnitude in the second tubing segment 125b. The pressure sensor 140 may be, for example, a differential pressure sensor. The pressure sensor 140 may be configured to provide pressure signals to the microcontroller 150 based on pressure changes detected in the second tubing segment 125b. One port of the pressure sensor 140 may be open to the surrounding atmosphere. Another port may communicate with the second tubing segment 125b. Thus, in one aspect, the pressures detected can be the pressure differences between the ambient atmosphere and the interior of the second tubing segment 125b. In another aspect, pressure detected may be a magnitude of pressure in the interior of the second tubing segment 125b. In still yet another aspect, detected pressure detected may be performed over the duration of one or more time lapses.

The blood oxygen sensor 170 and the carbon dioxide sensor 180 may provide further accuracy in embodiments supplying oxygen to the user 99. The blood oxygen sensor 170 may be attached to an appropriate location on the user 99. For example, the blood oxygen sensor 170 may be positioned at a fingertip or an ear lobe of the user 99. The blood oxygen sensor 170 may be connected to the microcontroller 150 and configured to measure oxygen saturation (SPO₂), using pulse oximetry. SPO₂ data may be transmitted to the microcontroller 150 for use in calculating the amount of oxygen to supply the user, in combination with the inhalation pressures, during a breathing event. The carbon dioxide sensor 180 may be connected to the microcontroller 150 and configured to measure carbon dioxide present in the exhalation phase of the user 99. The amount of carbon dioxide present in the exhalation may be provided to the microcontroller 150 for determining an appropriate bolus of oxygen delivered to the user 99 in a subsequent inhalation phase.

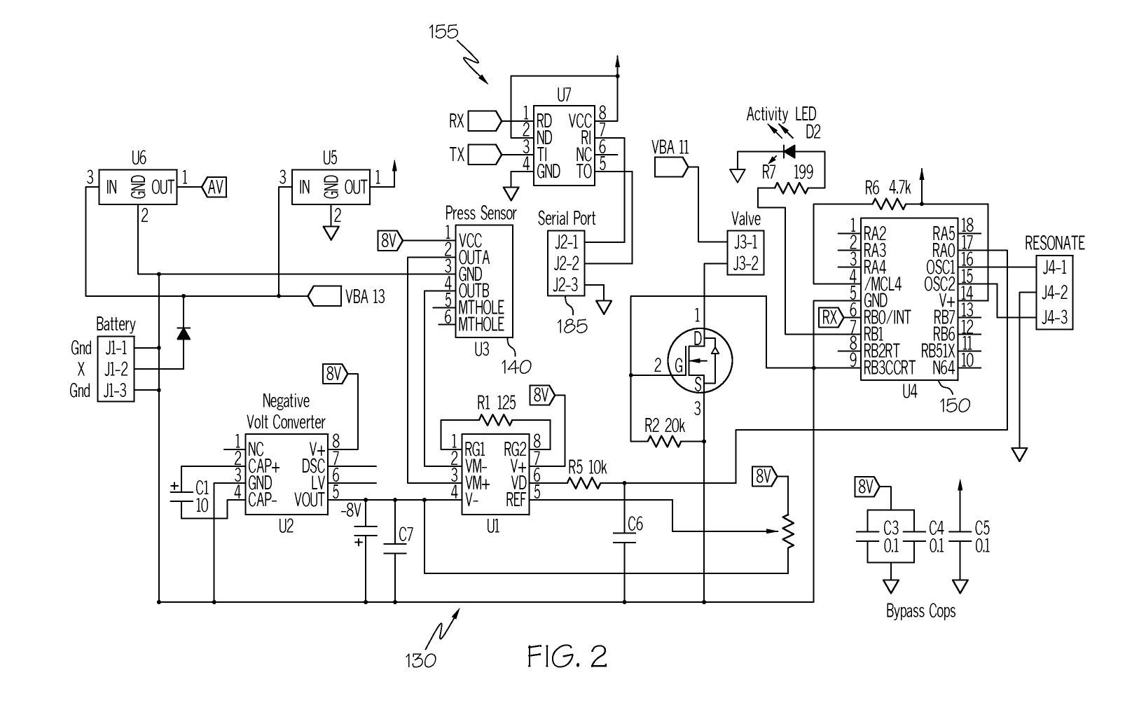

FIG. 2 shows an exemplary embodiment of a circuit schematic of the circuit board assembly 155. The circuit board assembly 155 shown is an embodiment that does not include the blood oxygen sensor 170 and the carbon dioxide sensor 180 of FIG. 1, but it will be understood that these two elements may be included or accommodated accordingly in embodiments that are configured for their use. It will also be understood that the support circuits 130 in this figure may include all of the features not designated by another reference number. The support circuits 130 may be configured to regulate power supplies on the circuit board assembly 155, to regulate amplifiers, to condition and effect accurate measurement of analog signals between the pressure sensor 140 and the microcontroller 150, to interface the communications port 185 to optional external equipment (for example, monitoring device/communications device 190 or other devices shown in FIG. 1), to provide alarm circuitry, and to provide other system monitoring circuits.

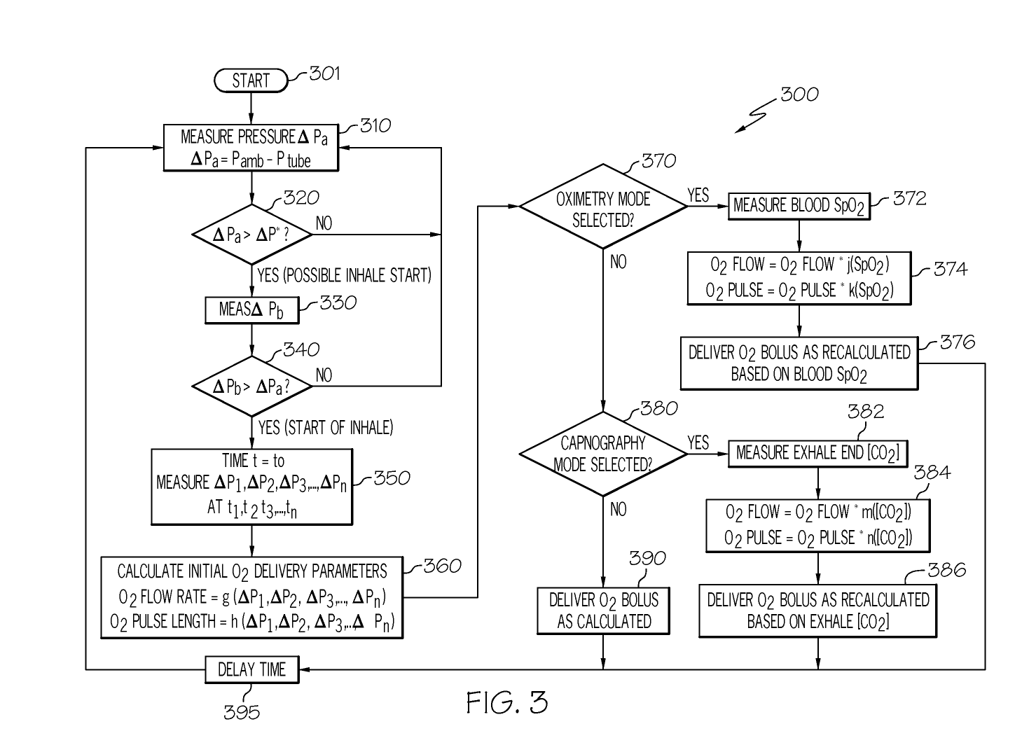

Referring to FIGS. 1 and 3, an exemplary method 300 of supplying fluid to a user 99 in a system 100 is shown. A continuous pressure measurement 310 in the second tubing segment 125b may be performed. A first pressure measurement (ΔPₐ) may be based on a difference between an ambient pressure (P_{amb}) and a pressure (P_{tube}) 1 in the second tubing segment 125b. The ambient pressure (P_{amb}) may be, for example, pressure detected exterior of the second tubing segment 125b. The microcontroller 150 may determine 320 if the measured pressure (ΔPₐ) is greater than a threshold pressure P*. If not, the method 300 returns to continuously measuring pressure 310. If yes, a second pressure measurement (ΔP_{b}) 330 may be performed.

The start of a breathing event may be detected 340, based on the microcontroller 150 detecting that a pressure drop in the second tubing segment 125b has occurred from the user 99 beginning an inhalation. The pressure drop may be based on the second pressure measurement (ΔP_{b}) is greater than the first pressure measurement (ΔPₐ). The microcontroller 150 may analyze 350 a plurality of additional pressure signals from the pressure sensor 140. For example, the microcontroller may analyze a plurality of pressure differential measurements (ΔP₁, ΔP₂, ΔP₃, . . . , ΔPₙ) between the ambient environment and the pressure in the second tubing segment 125b.

Pressure signals may also be analyzed over a predetermined time span at a plurality of times (t₁, t₂, t₃, . . . , tₙ); for example, 30 milliseconds from the start of the breathing event. An initial amount of oxygen may be determined 360. In exemplary embodiments providing continuous fluid flow, the amount of oxygen for delivery may be based on a function g of the plurality of pressure differential measurements (ΔP₁, ΔP₂, ΔP₃, . . . , ΔPₙ). For exemplary embodiments providing pulsed fluid flow, the amount of oxygen delivered may be based on a function h of the plurality of pressure differential measurements (ΔP₁, ΔP₂, ΔP₃, . . . , ΔPₙ). In one aspect, the determined amount of fluid may be delivered 350 to the user 99 during the detected breathing event, early during inhalation.

For embodiments utilizing a blood oxygen measurement 370, the blood oxygen sensor 170 may measure 372 oximetry data. The microcontroller 150 may determine 374 how much more or less of the initially determined 360 oxygen, either continuous flow or pulsed flow for example, should be provided to the user 99 based on the measured 372 oximetry data. Inclusion of a physiological measurement such as blood oxygen may allow a closed-loop mode operation in the system 100. Thus, an optimum amount of oxygen may be based on the measured pressure in the system 100 and may take into account the measured blood oxygen and modify for delivery 376 to the user 99 the calculated bolus size accordingly, to keep the actual blood oxygen within the physiologically appropriate range. The extent of the closed-loop moderation could range from no supplemental oxygen being delivered if the user's blood oxygen is already being maintained within physiologically appropriate limits, to extra, additional oxygen delivered under conditions where the user's blood oxygen may be falling. This type of operation provides optimization because oxygen is conserved at times where it is not needed, while being able to provide additional oxygen should the user's measured blood oxygen indicate additional need.

For exemplary embodiments using a capnography mode 380, the carbon dioxide detector 180 may detect 382 how much carbon dioxide is present in an exhalation of the user 99. The detection 382 of the amount of carbon dioxide detected may be used by the microcontroller 150 in determining 384 how much fluid, (either continuous flow or pulsed) should be provided 386 during a subsequent inhalation or detected breathing event.

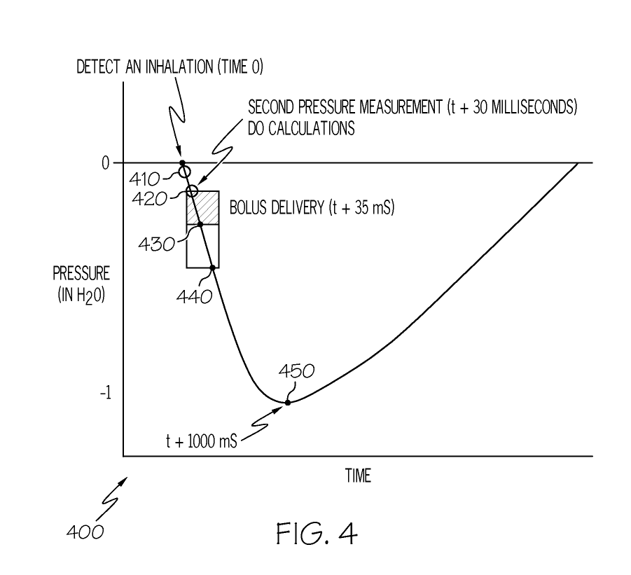

Referring now to FIG. 4, a breathing event timeline plot 400 is shown according to an exemplary embodiment of the present invention. A pressure sensor may measure pressure in tubing. A user inhaling fluid through tubing may create a drop in pressure in the tubing. It may be appreciated that aspects of the present invention provide detection and calculation of fluid needs and provide a required amount of fluid early in the inhalation phase of a breath cycle. The following numbered points represent events during changes in pressure of a breathing event. At point 410, a threshold pressure change may be represented. A threshold pressure change may, for example, be approximately 0.08 inches of water. The detection of the threshold pressure change may mark the detection of the start of an inhalation (breathing event). A subsequent pressure measurement(s) may be taken over a predetermined time lapse from point 410 to point 420. Inhalation pressure characteristics may be determined based on pressure measured at point 410 and any subsequent pressure signals measured between point 410 and point 420, including any at point 420. The inhalation pressure characteristics thus measured may be used to determine at point 430, an optimum fluid amount for delivery to the user over approximately the next 5 milliseconds. After the time lapse determining fluid amount, at point 440, the determined amount of fluid may be delivered through the system to the user approximately 35 to 50 milliseconds after the detection of the breathing event. At point 450, the user reaches the peak of inhalation (illustrated in this depiction as the lowest point of pressure in the tubing), after approximately 1000 milliseconds from the start of the breathing event. It will be understood that the shape, amplitude and time lapse of the pressure trajectory between the start of a breathing event and peak inhalation may vary from breath to breath depending on several factors including the state of exertion of the user.

It should be understood, of course, that the foregoing relates to exemplary embodiments of the invention and that modifications may be made without departing from the spirit and scope of the invention as set forth in the following claims.

We claim:

1. A system of providing fluid to a user, comprising:
distensible tubing;
a flow controller coupled to the tubing and configured to control a flow of fluid through the tubing; and
a fluid flow adjustment module connected to the tubing and the flow controller, the module being configured to measure pressure changes in the tubing during a single inhalation and control the flow controller to provide an optimum amount of the fluid through the tubing based on the measured pressure changes during the inhalation.

2. The system of claim 1, wherein the fluid flow adjustment module includes a microcontroller configured to determine the optimum amount of the fluid to be delivered through the flow controller based on the measured pressure changes during the inhalation.

3. The system of claim 2, wherein the optimum amount of fluid is a bolus of oxygen.

4. The system of claim 1, wherein the tubing is a cannula.

5. A system of providing oxygen to a user, comprising:
distensible tubing connected between an oxygen source and the user to provide an amount of oxygen to the user;
a flow controller coupled to the tubing and configured to control the amount of oxygen through the tubing;
a pressure sensor connected to the tubing between the flow controller and the user; and
a microcontroller coupled to the pressure sensor, the microcontroller being configured to:
receive pressure signals provided by the pressure sensor, wherein the pressure signals are detected from differential pressure in the tubing,
detect the start of a breathing event from the user based on a first pressure signal,
determine the amount of oxygen needed by the user based on a second pressure signal, and
control the flow controller to adjust the amount of oxygen flow to the user based on the second pressure signal.

6. The system of claim 5, wherein the microcontroller is configured to control the flow controller to deliver the determined amount of oxygen during the breathing event.

7. The system of claim 5, including a blood oxygen sensor connected to the microcontroller and adapted to be attached to the user, the microcontroller being configured to determine the amount of oxygen needed based on measurements taken by the blood oxygen sensor.

8. The system of claim 5, including a bypass valve connected between the oxygen source and the user, the bypass valve disposed to allow continuous oxygen flow to the user when the system malfunctions.

9. The system of claim 5, including a carbon dioxide sensor connected to the microcontroller, the microcontroller being configured to determine, based on measurements taken by the carbon dioxide sensor, a second amount of oxygen to be delivered during an inhalation subsequent occurring subsequently to the breathing event.

10. A method of providing oxygen to a user, including:
detecting the start of a first breathing event in tubing connected to the user;
analyzing a magnitude of pressure change in the tubing during a predetermined time frame;
determining an amount of oxygen needed by the user during the first breathing event based on the magnitude of pressure change analyzed; and
supplying the determined amount of oxygen to the user.

11. The method of claim 10, wherein detecting the start of the first breathing event includes detecting a pressure drop in the tubing greater than a predetermined threshold pressure.

12. The method of claim 10, wherein the pressure change occurs during an inhalation phase of the breathing event.

13. The method of claim 10, wherein supplying the determined amount of oxygen to the user is performed early in the inhalation phase of the first breathing event.

14. The method of claim 10, wherein the determined amount of oxygen to the user is performed within a predetermined time from the detection of the start of the breathing event.

15. The method of claim 10, wherein the analyzed magnitude of pressure change is based on a difference of ambient pressure and a pressure in the tubing.

16. The method of claim 10, including measuring blood oxygen levels in the user, wherein determining the amount of oxygen needed is based in part on the measured blood oxygen levels.

17. The method of claim 10, including:
measuring carbon dioxide levels of the user during an exhalation phase of the first breathing event; and
determining the amount of oxygen to be supplied to the user during the inhalation phase of a second breathing event, based in part on the measured carbon dioxide levels, wherein the second breathing event occurs after the exhalation phase of the first breathing event.
